(19) United States
(12) Patent Application Publication
(10) Pub. No.: US 2006/0246603 A1
(43) Pub. Date: Nov. 2, 2006

(54) Generation of chemiluminescence by hydrogen

(76) Inventors: Ursula Giesen, Weilheim (DE); Jurgen Heinze, Freiburg (DE); Kai Borgwarth, Freiburg (DE)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/525,883

(22) PCT Filed: Aug. 21, 2003
(86) PCT No.: PCT/EP03/09299

§ 371 Date: Nov. 4, 2005

(30) Foreign Application Priority Data
Aug. 26, 2002 (DE) 102 39 098.3

(51) Int. Cl. G01N 33/543; G01N 33/20

(52) U.S. Cl. 436/518; 436/84

(57) ABSTRACT

The invention concerns a method for detecting an analyte in a sample using a luminescent metal complex as a labelling group and a device that is suitable therefor.

The invention describes improved measuring cells having an electrode arrangement according to the invention.

Devices and methods for carrying out electrochemiluminescence measurements are known in the prior art. Such a device is described in detail in the patent application WO 89/10551. The apparatus comprises a measuring cell which has an inlet and an outlet and several electrodes located in its interior. The two electrodes of different polarity which are necessary for ECL measurements are juxtaposed in one plane. An optical window through which the optical radiation can be detected, is located within the measuring cell opposite to this plane. The said patent application primarily concerns the electrochemical conditioning of the electrodes prior to a measurement in order to create identical initial conditions for successive measurements. Reference is herewith made to the patent application WO 89/10551 in its entirety. Furthermore the document WO 89/10552 discloses an electrochemiluminescence measuring cell in which the working electrode is located in the interior of the cell volume. This arrangement results in special requirements for the electrode which has to be light-permeable and electrically conductive under the conditions described in the document. The working electrode has to fulfil the same requirements in the document EP 0 525 212 which also discloses a method for measuring electrochemiluminescence.

The object of the invention was to improve existing measuring cells for electrochemiluminescence measurements with respect to sensitivity, reproducibility and long-term stability.

According to the invention it was found that these objects can be achieved by selecting a cell where the electrodes are arranged such that the working electrode and counter electrode are not in one plane and that part of the volume of the measuring cell is enclosed between the two electrodes.

The invention concerns a device for generating optically detectable signals by applying electrical potentials to sample liquids comprising

a measuring cell for receiving sample liquids which has at least two openings for delivering and discharging liquids, a voltage source whose voltage is controllable, at least one planar working electrode which is adjacent to an inner wall of the measuring cell and is connected to a first pole of the voltage source, at least one counter electrode which is located within the measuring cell and is connected to a second pole of the voltage source, an optical window which is located in a wall of the measuring cell, a magnet which can be used to deposit microparticles on the working electrode, characterized in that the at least one counter electrode is at least partially disposed in the optical path between the optical window and the at least one working electrode such that the working and counter electrode are not in one plane and that part of the volume of the interior space of the cell is located between them and the optical signal is screened by the counter electrode.

A device according to the invention for generating optically detectable signals has a measuring chamber with preferably two openings for delivering and discharging liquids.

The measuring cell can be manufactured from a single piece or be made of different interconnected parts. Materials known in the prior art come into consideration as materials for the measuring cell such as plastics, glass and metals. If a cell is used which is composed of several parts, these parts can for example be glued, screwed, riveted or welded.

The interior space of the measuring cell is preferably shaped in such a manner that it is flushed as completely as possible when liquid is passed through the openings. Hence preferred embodiments of the interior space do not have recesses or such like. The interior space preferably has an elongate shape which is flattened in a direction perpendicular to the direction of flow.

At least one working electrode and one counter electrode are located in the interior space of the measuring cell. The electrodes are preferably attached to the inner walls of the measuring cell. They can for example be attached by gluing, melting or pressing.

The at least one working electrode has a planar shape and is electrically connected to a controllable voltage source. Suitable materials for the working electrode are noble metals such as gold, silver, platinum, palladium, ruthenium, osmium, tungsten or mixtures of these metals. Gold and platinum are particularly preferred electrode materials.

The at least one counter electrode can consist of a single piece or be composed of several parts which are connected to one another in an electrically conductive manner. The counter electrode preferably consists of two or more strips which are arranged opposite to the working electrode. Suitable materials for the counter electrodes correspond to those that are used for the working electrode. According to the invention the working and counter electrodes are not disposed in one plane but opposite to each other such that a part of the cell interior is located between them. The at least one working electrode and the at least one counter electrode are preferably disposed in parallel planes between which a part of the cell interior space is located.

For both the working electrode and the counter electrode it has proven to be advantageous when the surfaces reflect radiation and in particular the radiation generated in the ECL process. The areas of the electrodes are in the range of mm² to cm². The working electrode is usually square or rectangular with edge lengths in a ratio of 1 to about 3. Counter electrodes usually have a smaller area and preferably have an elongate shape with edge lengths in a ratio of 3 to about 30. Both the working and counter electrode are preferably thin plates with thicknesses ranging from a few tenths of millimetres to a few millimetres.

Net-like arrangements or plates with recesses are for example also possible for the counter electrode.

The working electrode is connected to the first pole of a controllable voltage source and the counter electrode is connected to a second pole of the same voltage source. The voltage source can be configured such that the voltage can be manually regulated. However, the voltage source is preferably controlled by a microprocessor or another control device. The voltage source must be able to provide voltages up to a few volts. According to the invention it is possible to control the magnitude as well as the time course of the voltage. In many process cycles the working electrode is the anode and the counter electrode is the cathode, but it is also possible to change the polarity of the electrodes.

The voltage to be supplied by the voltage source is mainly, but not exclusively, determined by the redox systems used in the process, and especially on the oxidation potential of the ECL label. It has turned out that the electrode arrangement according to the invention requires lower voltages to generate electrochemiluminescence than is the case with previously known measuring cells using the same sample liquids and redox systems. Some of the advantages mentioned above are a result of this lower working voltage compared to the prior art. It has turned out that the luminescence signal occurs much faster after applying the working voltage with a measuring cell according to the invention than is the case with previously known measuring cells. Moreover the measuring signal of a measuring cell according to the invention can be evaluated more accurately since it has a more sharply defined maximum.

A window is located in at least one wall of the measuring cell through which electro-magnetic radiation from at least one of the ranges infrared, visible and ultraviolet can pass. The entire cell is preferably made of a material which is permeable to at least one of the aforementioned types of radiation. A particularly suitable material for the optical window and/or the entire cell is polymethylmethacrylate.

It has also proven to be particularly advantageous according to the invention for the at least one counter electrode to be located on the inner side of the optical window.

The optical window is arranged such that the radiation which is generated on the working electrode can at least partially emerge from the measuring cell through the window. Radiation which emerges through the optical window can be detected by means of a detector. Suitable detectors are for example photomultipliers and semiconductor detectors. In a device according to the invention at least part of the at least one counter electrode is located in the optical path of radiation which is generated in the vicinity of the working electrode and emerges from the measuring cell through the optical window. Since at least part of the counter electrode is located in the path of the radiation emitted by the ECL label it was initially assumed that the radiation would be shielded and thus lead to a reduction in the signal. When compared to measuring cells where the counter electrode is not located in the optical path, it was surprisingly found that the electrode arrangement according to the invention results in stronger signals.

In a preferred embodiment a magnet outside the measuring cell can be moved onto the wall on which the working electrode is located inside the measuring cell. Electrical and permanent magnets can be used as the magnet. Permanent magnets are preferred since they do not generate heat during operation. In an automated apparatus the magnet can be moved towards or away from the working electrode by means of a spindle drive or a lever arm.

It is advantageous for the interior space of the measuring cell to be electrochemically connected to a reference cell. The electrochemical coupling can for example be achieved by means of a liquid-filled capillary gap or a frit. It is essential for an electrochemical coupling that charged particles can be exchanged between the inside of the cell and the reference electrode but that the amount exchanged is limited in order to substantially prevent contamination of the liquid in the cell interior. Electrodes known in the prior art such as an Ag/AgCl electrode or a calomel electrode are suitable as reference electrodes.

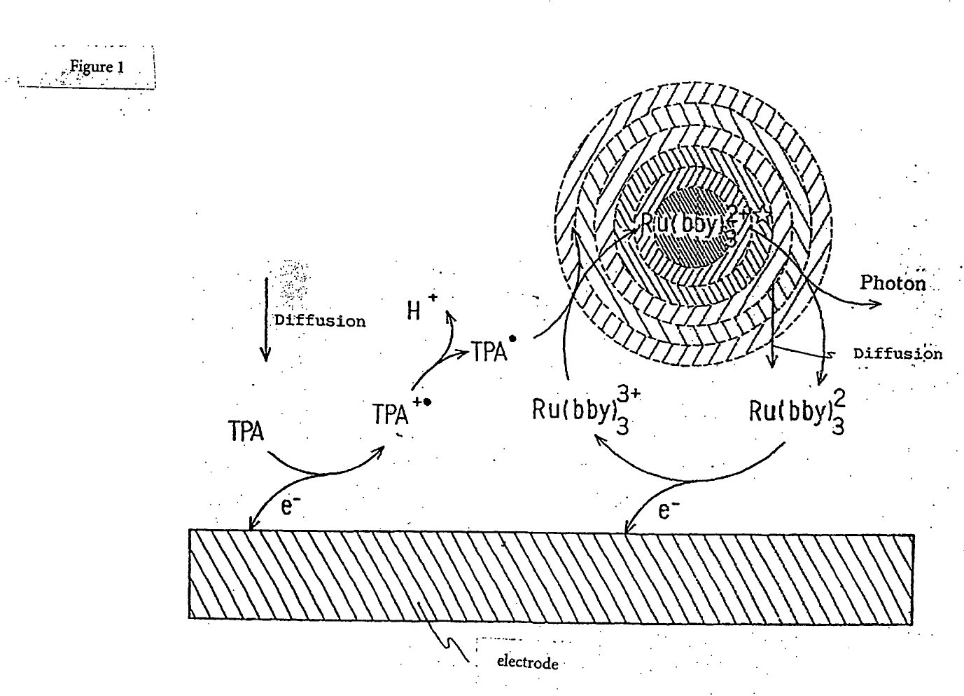

A device according to the invention was designed to measure electrochemiluminescent phenomena. During an ECL process chemical species are generated on the surface of an electrode which emit electromagnetic radiation in the infrared, visible or ultraviolet range. The electrochemical reactions which occur in such a process are generally described in the aforementioned patent application WO 89/10551. FIG. 1 is an example showing possible electrode reactions. Tripropylamine (TPA) is oxidized at the electrode and subsequently cleaves off a proton. In a second cycle Ru(bpy)3²⁺ is oxidized to Ru(bpy)3³⁺. The TPA radical and the oxidized ruthenium complex react to form another ruthenium complex which is converted into Ru(bpy)3²⁺ while emitting radiation at 620 nm. This and other electrochemiluminescent systems known in the prior art can be used to carry out chemical and immunological analyses.

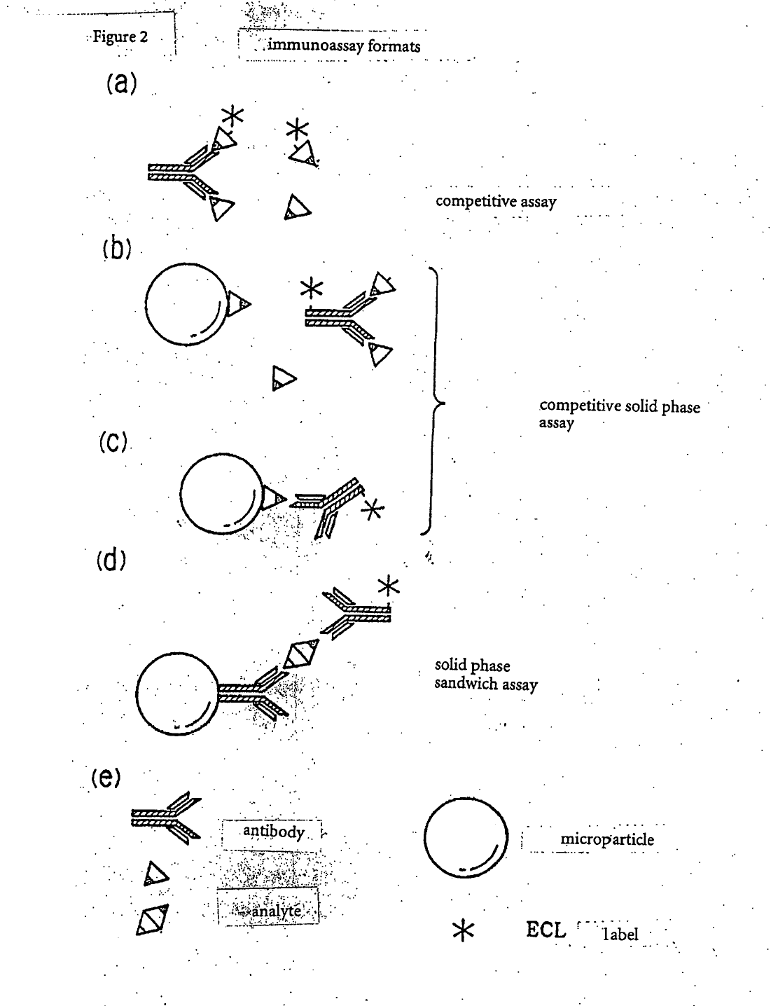

FIG. 2 shows a diagram of three different analytical methods that can be performed with the ECL technology. The format in FIG. 2A is based on the fact that the ECL signals of ECL labels that are bound to an antibody differ from those that are free in solution. FIG. 2B shows a format where a microparticle which is provided with an antigen competes with the analyte for an antibody which is provided with an ECL label. If there is no separation, the basis of the ECL measurement is that the ECL label will have different signal intensities depending on whether the ECL label is bound to a microparticle or to an analyte molecule. The reason for this behaviour has not been completely clarified but it is certain that the different diffusion properties of these species play a role in the electrode reaction. If a separation step is included in the format shown in FIG. 2B, the species that are bound to the microparticles can for example be separated by applying magnetic forces provided the microparticles have ferromagnetic properties.

FIG. 2C shows a third variant where the signal yield is directly proportional to the concentration of the analyte.

The invention also encompasses a method for generating optically detectable signals by applying electrical potentials to sample liquids containing microparticles using a measuring cell in which at least one planar working electrode which is adjacent to an inner wall of the measuring cell and at least one counter electrode are located and the measuring cell has an optical window, wherein the at least one counter electrode is at least partially disposed in the optical path between the optical window and the at least one working electrode such that the working and counter electrode are not located in one plane and that part of the volume of the interior space of the cell is located between them and the optical signal is screened by the counter electrode, comprising the steps
filling the measuring cell with liquid containing microparticles with electrochemiluminescent labels,
depositing microparticles on the working electrode with a magnet
applying a voltage profile to the at least one working electrode and to the at least one counter electrode opposite to the working electrode to generate electrochemiluminescent radiation,
detecting radiation which emerges through the optical window.

In this process a measuring cell is firstly filled with sample liquid. This can be achieved with a device according to the invention in which sample liquid is pumped into an opening in the measuring cell.

Sample liquid is understood to be a mixture of liquids comprising analyte solution, reagent solution and optionally auxiliary solutions.

The analyte solution, reagent solution and optionally auxiliary solutions can be introduced into the measuring cell either together or successively.

An analyte solution is understood as a solution, suspension or emulsion of analyte in a solvent such as water) acetonitrile, dimethylsulfoxide, dimethylformamide, N-methylpyrrolidine, tert-butyl alcohol or mixtures of these solvents.

Whole blood, serum, tissue fluid, saliva, urine etc. can for example be used as the analyte liquids. Analyses Lo be detected in these analyte liquids may for example be cells, subcellular particles, viruses, nucleic acids, proteins, peptides, hormones, pharmaceutical agents, organic molecules etc.

A reagent solution contains an ECL label i.e. usually an organometallic compound which emits electromagnetic radiation as a result of chemical and electrochemical reactions. The metal of the organometallic compound is preferably selected from the following group: ruthenium, osmium, rhenium, iridium, rhodium, platinum, palladium, molybdenum and tungsten. The ECL label may be bound to a whole cell, subcellular particles, viruses, fats, fatty acids, nucleic acids, polysaccharides, proteins, lipoproteins, lipopolysaccharides, glycoproteins, peptides, cellular metabolites, hormones, pharmaceutical agents and degradation products thereof, alkaloids, steroids, vitamins, amino acids, sugar, organic molecules, organometallic molecules, inorganic molecules, biotin, avidin or streptavidin.

The above-mentioned auxiliary solutions can for example include rinsing solutions which are suitable for cleaning the interior space of the measuring cell. Accordingly rinsing solutions may contain detergents, substances which decrease the surface tension, solvents for organic materials etc.

According to the invention it is preferable to rinse the cell with a cleaning solution or conditioning solution prior to the actual filling with sample liquid. A cleaning solution is understood to be any liquid which can be used to remove contamination and residues from the cell such as detergent solutions, solvents etc. Conditioning solutions are mainly used to put the electrodes in a defined oxidation and surface state. Hence conditioning solutions can contain oxidizing agents, reducing agents or surface-active substances.

After the cell has been filled with sample liquid, a voltage profile is applied to the at least one working electrode and the at least one counter electrode.

A voltage profile is understood to-be a time sequence of voltages of different magnitudes. In the simplest case a constant low voltage is for example applied to the electrodes and this is suddenly increased in order to initiate the electrode reaction necessary for the ECL reactions. Favourable voltage profiles are described for example in the patent application WO 90/11511.

According to the invention it is preferred that the measuring cell and the electrodes are cleaned and conditioned before the actual measuring step is carried out. The effect of the cleaning and/or conditioning solutions in these steps is enhanced by applying suitable voltage profiles to the electrodes.

The previously described procedure is suitable for a homogeneous assay i.e. an analysis where there is no separation of the complex formed of analyte and ECL label.

According to the invention it is preferable to separate the complexes-formed from the ECL label and analyte before activating the electrochemical luminescence. This is preferably achieved by either directly binding the ECL label to a ferromagnetic particle or by forming a sandwich complex with the analyte and an antigen which is in turn bound to a ferromagnetic microparticle. The complex containing the ferromagnetic microparticle can be deposited on the working electrode by a magnet while excess ECL label is washed away.

Electrochemiluminescent radiation is emitted shortly after applying a suitable voltage profile for generating an ECL signal. The emitted radiation can be detected by means of a detector and the analyte concentration can be derived from the intensity of the radiation.

Compared to the prior art, a device and process according to the invention have the advantage that a given electrochemiluminescent reaction can be generated with a lower voltage than is the case with prior devices. The redox potential for the redox system shown in FIG. 1 is approximately 1.1 V. Reliable measurements with the IGEN measuring cell could be carried out at 2.2 V. With the measuring cell of the invention it was possible to reduce the measuring voltage to 1.4 V.

An advantage of reducing the measuring voltage is that fewer interfering side reactions occur which improves the signal to noise ratio.

Moreover it has also turned out that the measured result obtained with a measuring cell according to the invention is less dependent on the distribution of the microparticles on the working electrode than with measuring cells of the prior art. This property of the measuring cells according to the invention increases the reproducibility of the analytical detection.

Another advantage of measuring cells according to the invention is their improved dynamics which enable more sensitive and more reliable measurements.

A device according to the invention and a process according to the invention are illustrated by the figures.

FIG. 1: Schematic representation of the processes occurring at the electrodes.

FIG. 2: Examples of immunological test formats that can be carried out with the measuring cell.

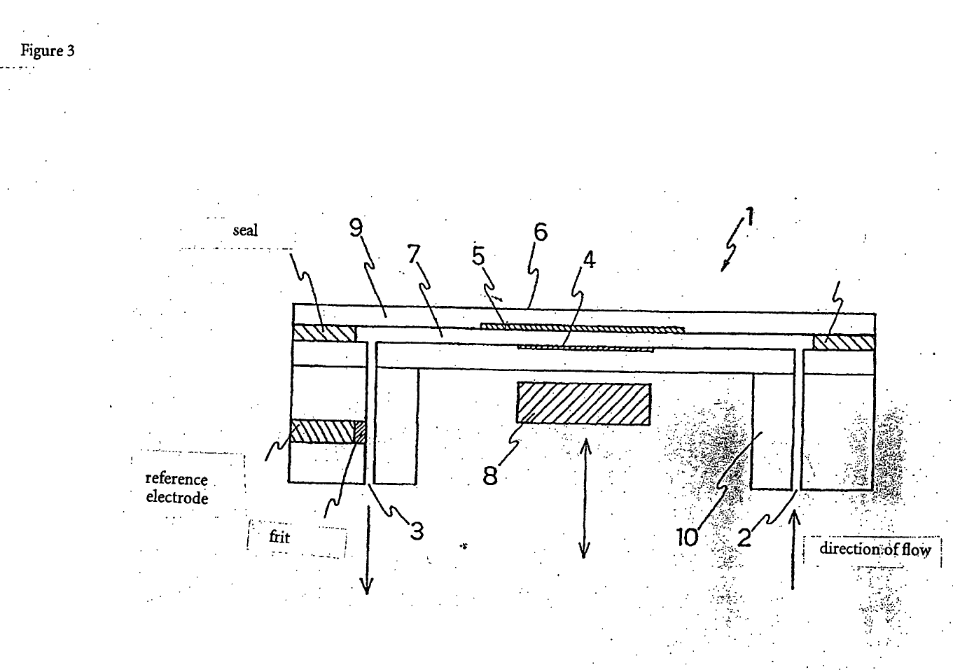

FIG. 3: Side view of the measuring cell.

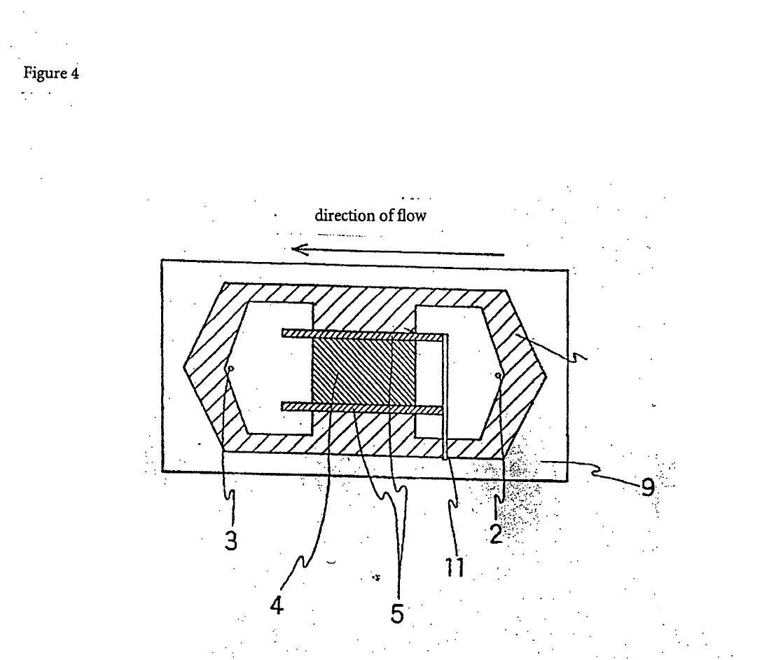

FIG. 4: Top view of the measuring cell,

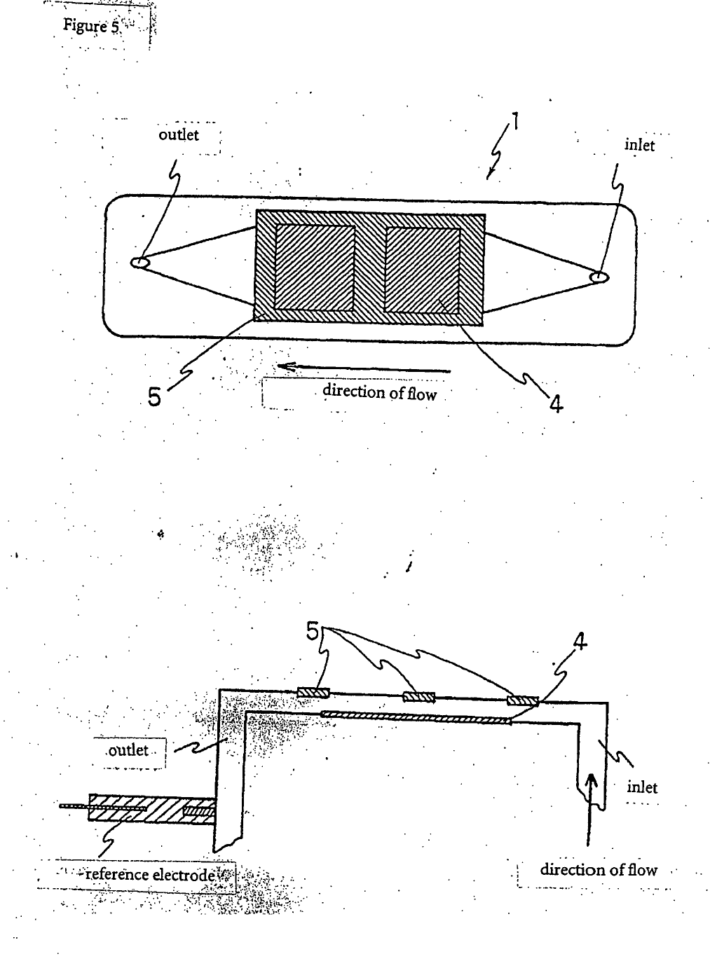

FIG. 5: Modified embodiment of a measuring cell in a side and top view

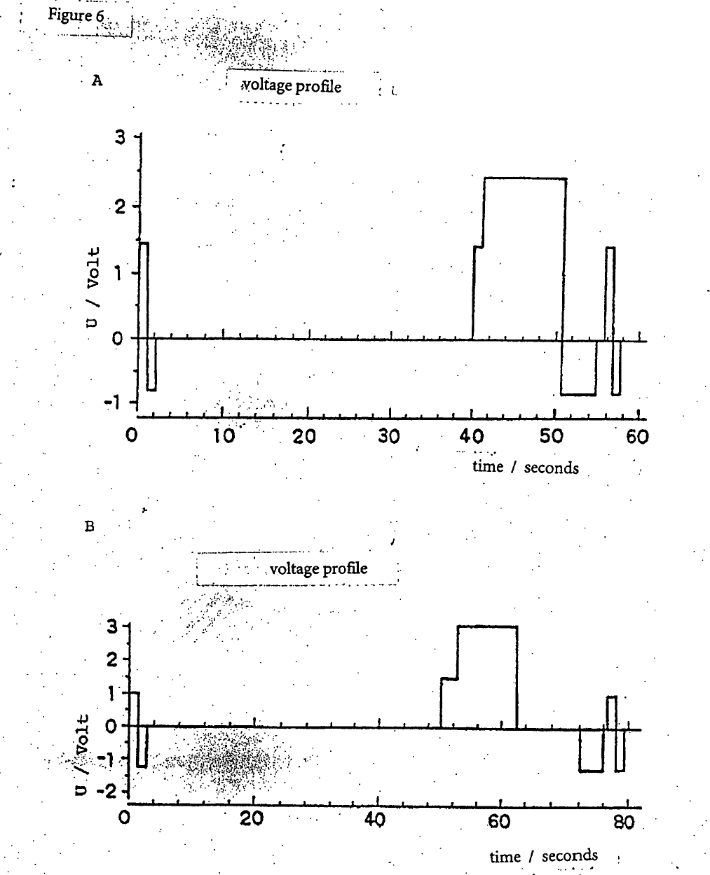

FIG. 6: Voltage time courses for operating measuring cells according to the invention.

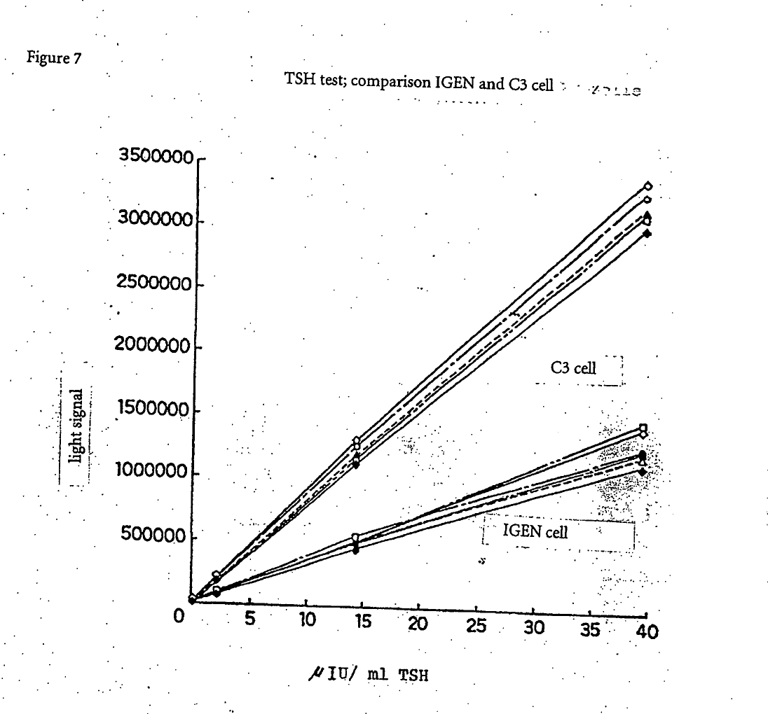

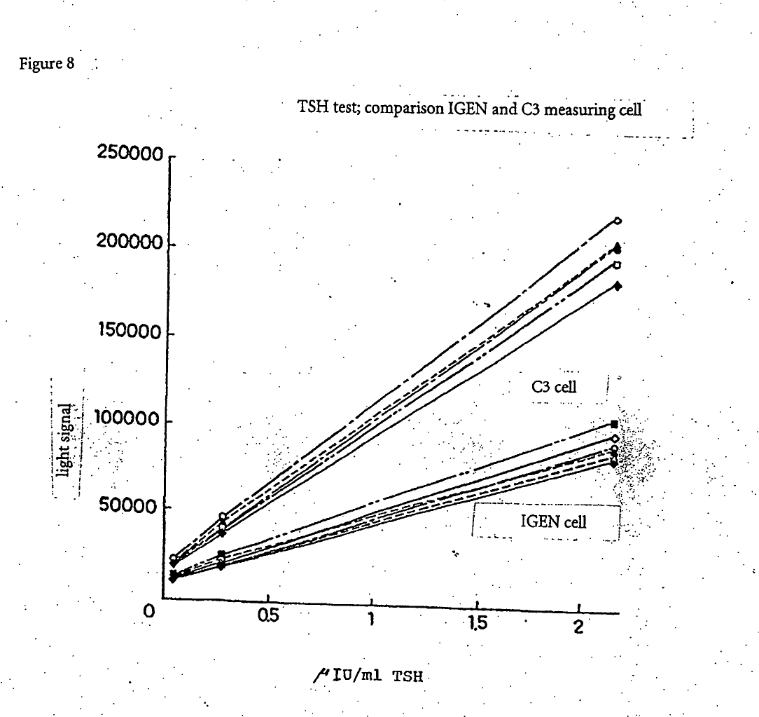

FIGS. 7, 8: Comparison of a measuring cell according to the invention (C3) with a known measuring cell from the prior art (IGEN).

FIG. 3 shows a side view of a device according to the invention. FIG. 4 shows a top view of the same device. The body of the measuring cell (1) consists of several polymethylmethacrylate components. The interior space of the cell (7) has an elongate flat shape. In FIG. 4 it can be seen that the inlet and outlet openings (2, 3) are arranged at an obtuse angle of a triangle. With this arrangement it is possible to avoid areas where any residual liquid may remain which is not part of the volume flow when liquid flows through the cell. A working electrode (4) which has a flat rectangular shape is located at the bottom (10) of the measuring cell. The cell shown in this figure has two counter electrodes (5) which have a flat, rod-like shape and are pressed into the upper portion of the measuring cell. The counter electrodes (5) are connected to a voltage source via a common line (11). The working electrode as well as the counter electrodes shown in this example are both made of platinum. An optical window (6) is located on the side of the measuring cell (1) which is opposite to the working electrode. In this example the cover (9) of the measuring cell is made of polymethylmethacrylate which renders it permeable to visible radiation. A magnet (8) which can be moved either towards or away from the working electrode is located below the working electrode (4).

FIG. 5 shows another embodiment of a measuring cell (1) according to the invention. The structure of this measuring cell corresponds essentially to the measuring cell shown in FIG. 3 and FIG. 4. One difference is the design of the counter electrode (5). This is arranged opposite to the working electrode (4) in accordance with the invention but has a rectangular shape with two rectangular recesses. Accordingly the counter electrode (5) has segments which are arranged parallel as well as perpendicular to the direction of flow in the measuring cell. FIG. 6A shows a voltage profile which is suitable for carrying out an ECL reaction with the species tripropylamine and Ru(bpy)3²⁺ in a measuring cell according to the invention. While the measuring cell is filled with a buffer solution, a voltage of +1.4 V is applied for one second and a voltage of −0.8 volt is applied for a further second. This conditioning phase serves to produce a defined surface state on the working electrode. During the following 39 seconds the measuring cell is filled with a mixture of analyte, reagent and buffer solution. A potential (e.g. 0 mV) at which no ECL reaction occurs but which maintains the electrodes in a reproducible state is applied to the electrodes. In order to generate the ECL signal, a potential of +1.4 volt is applied for one second. Subsequently the measuring cell is cleaned by-applying a voltage of +2.4 V for 9.5 seconds and −0.8 V for 4 seconds. The cleaning process terminates the measuring cycle. The described voltage profile can be used repeatedly for subsequent measurements.

FIG. 6B shows a voltage profile for operating a measuring cell according to the invention using a low measuring voltage. The following table provides further details on the voltage time course and the liquids introduced into the measuring cell during the individual phases:

| | Time/ | Volt- | Dura- | Amount of | | |
|---|---|---|---|---|---|---|
| Procedure | s | age/V | tion/s | liquid/time | | Type of Liquid |
| Conditioning of | 0 | 0 | | 167 | μl/s | Buffer solution |
| the measuring | 0 | 1 | 1 | | | |
| cell | 1 | 1 | | | | |
| | 1 | −1.2 | 1 | | | |
| | 2 | −1.2 | | | | |
| Filling of the | 2 | 0 | 1 | 152 | | Sample solution |
| measuring | 11.5 | 0 | 1.2 | 172 | | and reagent |
| cell | | | | | | solution |
| | 12.4 | 0 | 30 | | | |
| | 18.4 | 0 | 6 | 33 | | Buffer solution |
| | 44.6 | 0 | | 167 | | |
| Measurement | 50 | 0 | 1 | none | | |
| | 50 | 1.4 | | | | |
| Cleaning the | 53 | 1.4 | | 333 | | Cleaning solution |
| measuring cell | 53 | 3 | 9.5 | | | |
| | 63 | 3 | | | | |
| | 63 | 0 | | | | Buffer solution |
| | 72.5 | 0 | 4 | 167 | | |
| | 72.5 | −1.2 | | | | |
| | 76.5 | −1.2 | 1 | | | |
| Conditioning of | 76.5 | 0 | 1 | 167 | | Buffer solution |
| the measuring cell | 77.2 | 0 | | | | |
| | 77.2 | 1 | 1 | | | |
| | 78.2 | 1 | | | | |
| | 78.2 | −1.2 | | | | |
| | 79.2 | −1.2 | | | | |
| | 79.2 | 0 | | | | |
| | 79.9 | 0 | 0.3 | | | |

FIGS. 7 and 8 show a comparison of the measuring cell according to the invention (referred to as concept 3) with the IGEN measuring cell described in the patent application WO 89/10551 for two different analyte concentration ranges. The light signal plotted on the ordinate in arbitrary units was obtained with a photomultiplier of the Origen 1.0 instrument manufactured by the IGEN Company. The abscissa of the figures shows the concentration of TSH (thyroid stimulating hormone) in the sample.

It can be seen from the figures that the dynamics of the calibration curve is considerably higher when concept 3 cells are used which increases the sensitivity of the test.

The measurements were carried out by incubating 250 μl of the following mixture

50 μl buffer solution IP

50 μl bead suspension

50 μl biotinylated antibody (R1)

50 ml ruthenylated antibody (R2)

50 μl analyte

for 16 minutes at room temperature and then pumping 150 μl of the incubation mixture into the measuring cell where the beads were captured on the working electrode with the aid of a magnet and washed.

The composition of the solutions used is as follows:

Buffer Solution IP:

50 mM Tris pH 8.0

0.1% CAA

0.01% MIT

0.2% Thesit

5% BSA 1

1% B-IgG

Biotinylated Antibody (R1)

3.0 μg/ml MAD <TSH> M1.20-IgG biotin
(Boehringer Mannheim Catalogue No. 1352547)

500 μg/ml MAB <-> IgG
(Boehringer Mannheim Catalogue No. 1522558).

Ruthenylated Antibody (R2)

1.2 [2g/ml MAB <TSH> MAB-F(ab′)₂-BPRu

Bead Suspension

600 μg/ml M280 beads from the Dynal International Company (Oslo, Norway) was suspended in the buffer.

### LIST OF REFERENCE NUMERALS

(1) measuring cell

(2) inlet opening

(3) outlet opening

(4) working electrode

(5) counter electrode

(6) optical window

(7) interior space of the measuring cell

(8) magnet

(9) cover of the measuring cell

(10) bottom of the measuring cell

(11) line

1. Device for generating optically detectable signals by applying electrical potentials to sample liquids containing microparticles said device comprising
a) a measuring cell (1) for receiving sample liquids which has at least two openings (2, 3) for delivering and discharging liquids,
b) a voltage source whose voltage is controllable,
c) at least one planar working electrode (4) which is adjacent to an inner wall of the measuring cell and is connected to a first pole of the voltage source,
d) at least one counter electrode (5) which is located within the measuring cell and is connected to a second pole of the voltage source,
e) an optical window (6) which is located in a wall of the measuring cell,
f) a magnet which can be used to deposit microparticles on the working electrode wherein
the at least one counter electrode (5) is at least partially disposed in the optical path between the optical window (6) and the at least one working electrode (4) such that the working and counter electrode are not in one plane and that part of the volume of the interior space of the cell is located between them and the optical signal is screened by the counter electrode.

2. Device as claimed in claim 1, wherein the at least one working electrode (4) has a surface which is disposed parallel to the optical window (6).

3. Device as claimed in claim 1 or 2, wherein the at least one working electrode (4) is essentially composed of gold or platinum.

4. Device as claimed in claim 1, 2 or 3, wherein the at least one working electrode (4) at least partially reflects optical radiation.

5. Device as claimed in claim 1, wherein the radiation generated by the counter electrode is at least partially reflected.

6. Device as claimed in claim 1, wherein a reference cell is electrochemically coupled to the interior space of the measuring cell (1).

7. Device as claimed in claim 1, wherein the magnet for depositing the microparticles is disposed on the outside of the wall that is adjacent to the working electrode.

8. Device as claimed in claim 1 or 7, which comprises a-device for moving the said magnet towards and away from the said cell wall.

9. Device as claimed in claim 1, wherein two or several counter electrodes (5) are present which have a flat, rod-like shape.

10. Device as claimed in claim 1 or 9, wherein the direction of flow of the sample liquid when the cell is filled or emptied is parallel to the longitudinal axis of the at least one counter electrode (5).

11. Device as claimed in claim 1, which comprises a detector which detects radiation emerging from the optical window (6).

12. Process for generating optically detectable signals by applying electrical potentials to sample liquids containing microparticles using a measuring cell (1) in which at least one planar working electrode (4) which is adjacent to an inner wall of the measuring cell and at least one counter electrode (5) are located and the measuring cell has an optical window (6), wherein the at least one counter electrode is at least partially disposed in the optical path between the optical window and the at least one working electrode such that the working and counter electrode are not located in one plane and that part of the volume of the interior space of the cell is located between them and the optical signal is screened by the counter electrode, comprising the steps
a) filling the measuring cell (1) with liquid containing microparticles with electrochemiluminescent labels,
b) depositing microparticles on the working electrode with a magnet
c) applying a voltage profile to the at least one working electrode (4) and to the at least one counter electrode (5) opposite to the working electrode (4) to generate electrochemiluminescent radiation,
d) detecting radiation which emerges through the optical window (6).

13. Process as claimed in claim 12, wherein in step b) a magnet is moved towards the measuring cell (1).

14. Process as claimed in claim 12, wherein before step a) the measuring cell (1) is cleaned and the working electrode (4) and counter electrode (5) are prepared by applying a voltage profile.

15. Process as claimed in claim 14, wherein the cleaning and/or preparation are carried out in the presence of cleaning and/or conditioning solutions.

16. Process as claimed in claim 12 or 13, wherein the measuring cell is treated with a washing solution after step a).
